(19) United States
(12) Patent Application Publication
(10) Pub. No.: US 2008/0286851 A1
(43) Pub. Date: Nov. 20, 2008

(54) Large-scale photo-bioreactor using flexible materials, large bubble generator, and unfurling site set up method

(75) Inventor: Norman M. Whitton, Houston, TX (US)

Correspondence Address: ARNOLD & KNOBLOCH, L.L.P., 2401 FOUNTAIN VIEW DRIVE, SUITE 630, HOUSTON, TX, 77057, US

(73) Assignee: Sunrise Ridge Holdings Inc.

(21) Appl. No.: 11/803,298

(22) Filed: May 14, 2007

(51) Int. Cl. C12N 1/00; B21D 39/00; B23P 19/00; B23P 19/02; C12M 1/00

(52) U.S. Cl. 435/243; 29/426.4; 29/428; 29/700; 435/292.1

(57) ABSTRACT

A closed photo-bioreactor, which in at least one example comprises a plurality of flexible, repeating, substantially enclosed, parallel chambers flexibly connected along their lengths, where a liquid growth media is substantially still without the need for turbulent mixing of the bulk liquid. In many examples, each is connected into integrated, flexible pipelines that serve to supply gas to the chambers, to vent gas from the chambers, and to fill and drain the individual photo-bioreactor chambers of their liquid contents. In some installations, a bioreactor will be rolled up using, for example, a long rod as a spool, for storage and transportation. Some examples will be manually unfurled and positioned on an angled site including, for example, an earthen berm. In many embodiments, a photo-bioreactor will be manufactured from thin plastics using low cost manufacturing techniques. In at least one example, a photo bioreactor is described in which bubbles with a substantially non-convex shape are introduced to mix the liquid contents.

### BACKGROUND

1. Field of the Invention

This invention relates generally to the field of photo-bioreactors, and more particularly to the field of closed photo-bioreactors designed to use solar energy to grow photo-synthetic microorganisms or photo-microorganisms at a high yield, on a large scale and in a cost-effective manner.

2. Background of the Invention

Photo-microorganisms may be used as raw materials to produce oil, protein-enriched animal feeds, human foods, dyes, and as a means of reducing pollutants. Algae—one type of photo-microorganism—can provide vegetable oils suitable to produce biofuels with much higher oil yields than terrestrial crops, such as oil palm, coconut, canola or soybean. Oil production from certain microalgae species, for example Botryococcus_{—}braunii, may be as high as seventy five percent (75%) of plant mass, which represents a much more efficient conversion rate for solar energy to fuels. Some species of algae, such as Spirulina, can also produce human foods. Others can produce animal feed components, such as proteins, or specialty bio-products such as astraxanthin, a pigment used in shrimp farms. Photo-microorganisms may also be used to reduce pollutants, such as nitrate or phosphate in water, or carbon dioxide in air.

Current technologies for growing photo-microorganisms on a large scale include both open-air system photo-bioreactors (“open photo-bioreactors”) and closed system photo-bioreactors (“closed photo-bioreactors”). Both types of photo-bioreactors provide an environment containing an aqueous media with nutrients for algae growth, and a source of light. Additional components may be installed or used to adjust environmental factors (e.g. pH, mixing, gas exchange, temperature, etc).

Open photo-bioreactors, such as ponds or open raceways, are characterized by large areas of water freely open to the atmosphere. This allows foreign photo-microorganism species and unwanted microorganism predators to contaminate the system and lower yields of the desired photo-microorganism. Open systems also experience surface effects, such as waves that reduce solar energy absorption and thus energy efficiency. Additionally, the large, uncontrolled water-air interface renders it very difficult to control and optimize temperature and gas compositions, which in turn result in lower yields.

Previous closed photo-bioreactors avoid the problems of open systems but typically require expensive construction methods for component parts and expensive and complex set up requirements, including rigid pipes and tubes, metal guides, supports and other intricate, unwieldy and inadaptable support infrastructure. These closed systems have been implemented variously, by Greenfuels Technologies, based on MIT designs, by Greenshift Corporation, based on work at the University of Ohio and in other work at the University of California, Arizona State University and in Italy, Israel and Japan.

Examples include a closed photo-bioreactor that avoids the problems common to open systems while at the same time offering an alternative to the costly and inflexible structures associated with currently-available closed photo-bioreactor systems. Both the manufacture and deployment of such a closed photo-bioreactor affords substantial cost and convenience advantages over other closed photo-bioreactor systems.

An example uses large, intermittent bubbles to achieve mixing in a still or slow moving liquid containing the photo-microorganism. As a result, it may operate at a low fluid pressure, typically equivalent to the static head of the liquid. In turn, this permits the use of thin, flexible materials of construction that are low cost and easily deployed.

### SUMMARY

At least one example of a closed photo-bioreactor comprises a plurality of flexible, repeating, substantially enclosed, parallel chambers flexibly connected along their lengths and each preconnected, at the time of manufacture, to integrated, flexible pipelines that serve to supply gas to the chambers, to vent gas from the chambers and to fill (i.e. fill line), drain (i.e. drain line) or fill and drain (i.e. fill/drain line) the individual photo-bioreactor chambers of their liquid contents. This flexible yet integrated unit can be rolled up using, for example, a long rod as a spool, for storage and transportation.

In some deployments, once on location, the device can be deployed by unrolling and positioning it on, for example, an inclined earthen berm, giving each enclosed chamber an upper end that is raised with respect to the chamber's lower end. During the excavation of the berm (e.g., using a bulldozer or other earth moving equipment), the angle of the incline on the berm will be fixed in some example embodiments to optimize such factors as exposure to solar energy, gas flow within the chambers and drainage hydraulics.

After being unfurled and positioned on the berm, at least some examples require little else to be done before they are in full operational mode. Connections to sources of liquid and gas and to at least one liquid drain can be made quickly and simply. The photo-bioreactor will be, in some examples, filled with a water and growth media solution including an inoculation of the desired photo-microorganism.

A flexible gas supply line is part of the integrated unit in further examples, which includes an optional sparger and optional bubble generator located within each chamber. In some more specific examples, a gas supply line is connected at the time of manufacture to the lower end of each chamber to provide gas for respiration and conversion by the algae or other microorganisms meant to be grown by the photo-bioreactor. In situations where additional gas input into the individual closed chambers is desirable, examples of the invention will be constructed with additional, flexible integrated gas supply lines, optional spargers and optional bubble generators for generating large bubbles, pre-connected to the individual chambers at intermediate points along the chambers.

In many examples of the invention, the liquid contents of each chamber are still or very slow moving. Highly effective mixing will be achieved in some examples with low overall pressure and in the absence of turbulent bulk liquid flow by injecting a large bubble into the lower end of a chamber, and allowing the bubble to rise through a chamber. In further examples, large bubbles generate turbulent flow around their perimeters and displace the adjacent liquid, which mixes the liquid and the photo-microorganisms contained therein, as the large bubbles rise to the top of a chamber. In at least some examples, the large bubbles are characterized by a non-convex surface, typically in a trailing, bottom edge. The mixing induced by the generation of sufficiently large bubbles obviates the need to create high pressure to induce liquid circulation within each chamber. This in turn means that the chambers of this example can be made of lighter, more easily folded and transported, flexible material.

In one example, the large bubble generator consists of essentially the gas supply line and optionally a sparger. Pulsing the gas supply—pumping through large volumes of gas over a short period of time—will generate bubbles sufficiently sized to induce mixing in some examples. In other examples, a gas trap—in one configuration a flexible, hinged flap—will be used to generate sufficiently sized bubbles to generate the fluid flow required for mixing. In another example, a submerged gas chamber with a reverse siphon will be used to generate large bubbles. The latter two methods of generating a bubble within the chamber will be combined with a sparger in some examples. In some such embodiments, very small bubbles will be generated below or within the flexible flap or submerged gas chamber. This will enhance gas mass transfer (for instance carbon dioxide dissolution) into the liquid.

In some specific examples, an integrated unit includes a gas disengager or vent made with flexible materials, with an optional demisting arrangement, that is connected at the time of manufacture to the upper ends of each chamber. The demister separates the gas to be vented by the gas disengager or gas vent from the liquid that is to remain in the chamber.

In various examples, the integrated unit also includes one or more flexible, built-in lines that are preconnected upon manufacture to each chamber for the purpose of filling or draining the chambers' liquid media, nutrient and algae contents. A line that is constructed to be on the lower end of an integrated unit, in some examples, has the capability of both filling and draining the chambers (i.e. a fill/drain line).

By inclusion of one or more of the features described in this document, a system will be configured and put in working order at little material or labor cost (for example, by excavating an appropriately angled earthen berm, unfurling and positioning the integrated unit comprising the photo-bioreactor on the inclined plane of the berm with the gas vent line positioned to be elevated with respect to the gas supply line, connecting the preinstalled gas supply line that is part of the integrated photo-bioreactor to a source of carbon dioxide or other gas that can be used to support algae or other photo-microorganism growth, and connecting the integrated liquid fill line and/or integrated liquid fill/drain line to a supply of liquid medium, such as water and nutrients, needed to support microorganism growth) and connecting the liquid drain line and/or integrated liquid fill/drain line to a liquid drain. In some examples, the source of liquid and the liquid drain will be the same. Further advantages include easy and inexpensive preparation for relocation by, for example, disconnecting the unit's gas supply line and liquid fill and/or liquid fill/drain line from their respective sources, disconnecting the liquid drain and/or liquid fill/drain line from their respective liquid drains and rolling up the integrated unit, inclusive of the preconnected gas supply line, liquid fill line, liquid drain line, and/or liquid fill/drain line and gas vent or disengager line, for storage and transportation by using a long rod as a spool.

The versatility of various examples means that they will be set up for operation, and then disassembled, in any location where an angled resting place with proper exposure to sunlight can be arranged and the necessary gas and liquid lines connected to external sources of gas and liquid. This allows for deployment in locations that enable it to process industry generated waste, such as next to a carbon dioxide emitting power plant, or on a roof top near a chimney. In some such situations, the algae or other photo-microorganisms in the device will receive, through the gas supply line, waste carbon dioxide emitted by the facility and convert it into desired product, such as biofuel feedstock.

The above examples enjoy lower manufacturing costs over other closed photo-bioreactor systems. This is due in part to the fact that many examples can be constructed using low cost materials and techniques that allow photo-bioreactors to be made in high volumes but at low cost. The flexible plastic film (for example, 0.1 to 200 mil thick polyvinyl chloride, polyolefin, polyethylene terephthalate, polyimide, polyurethane or similar plastics) that comprises the walls of the individual chambers in some examples, the material connecting the chambers and the necessary gas and water lines are much less costly than rigid plastics, metals or glass. The connections among the various components of the device—at the points of connection between (i) the walls of the chambers, (ii) the flexible material connecting individual chambers along their lengths and the chambers themselves and (iii) between the integrated gas and water lines and the connections thereto on the chambers—may be joined using low cost joining methods such as plastic welding or adhesives.

In at least one example, the material comprising the integrated unit of the invention will be strengthened against punctures or tears with fibrous reinforcement during the manufacturing process. Fibrous geo-textile will be incorporated or embedded into the material of a photo-bioreactor. Alternatively, the fibrous geotextile will be laminated or glued to the outside of the photo-bioreactor. Including geotextile flaps that are flexibly connected, and extend beyond, the outside edges of the photo-bioreactor helps secure the photo-bioreactor to the angled earthen berm, or other angled site, and avoids the need, in the case of an earthen berm, to employ other erosion control methods on surrounding ground areas when installed.

At least one example comprises a photo-bioreactor comprising a plurality of substantially enclosed, flexibly connected, parallel chambers, at least some of said chambers comprising an upper end connected to at least one flexible gas vent line, a lower end connected to at least one flexible gas supply line, a lower end connected to at least one flexible liquid fill/drain line, and/or the upper end connected to a flexible liquid fill line and the lower end connected to a flexible liquid drain line, at least one transparent wall to each chamber; at least some of said plurality of chambers, said connections, said gas vent line, said gas supply line, said liquid fill line, said liquid drain line and/or said liquid fill/drain line being comprised of thin, flexible materials.

According to a further example, at least some of the thin, flexible materials comprise fibrous reinforcement.

Another example also includes a bubble generator for example, a gas trap, such as a flap, or at least one gas chamber with a reverse siphon disposed within at least some of the substantially enclosed, flexibly connected, parallel chambers.

According to a further example, at least some of the substantially enclosed, flexibly connected, parallel chambers will be connected to a gas supply line capable of generating variable pressure.

According to a further example, a method of setting up a photo-bioreactor is provided. In some examples, the method comprises the steps of preparing an angled site, unfurling said photo-bioreactor on said site, positioning said photo-bioreactor on said site so that there is an upper end that is elevated with respect to the lower end. In some further examples, said lower end is connected to a source of gas. In further examples the lower end is connected to a source of liquid and a liquid drain, and in other examples, the lower end is connected to a liquid drain and the upper end is connected to a source of liquid.

According to another example, a system for setting up a photo-bioreactor is provided. In at least one such example, the system comprises means for preparing an angled site, means for unfurling said photo-bioreactor, means for positioning said photo-bioreactor on said site so that there is an upper end that is elevated with respect to the lower end. In some such examples, a means for connecting the lower end to a source of gas is provided. In further examples, a means for connecting the lower end and/or upper end to a source of liquid is provided. In other examples, a means for connecting the lower end to a liquid drain is provided.

According to at least one further example, a method of handling a photo-bioreactor is provided comprising the step of rolling up said photo-bioreactor that includes in some examples at least one fold.

According to at least one further example, a system of handling a photo-bioreactor is provided comprising means for rolling up said photo-bioreactor that includes in some examples means for including at least one fold.

According to still another example, a method is provided of disassembling a photo-bioreactor comprising the steps of disconnecting input and/or output lines to said photo-bioreactor, and rolling up the photo-bioreactor that includes, in some examples, at least one fold.

According to another example, a system of disassembling a photo-bioreactor is provided comprising means for disconnecting any input or output lines to said photo-bioreactor and means for rolling up said photo-bioreactor that includes in some examples means for including at least one fold.

In still further examples, a method is provided of mixing photo-bioreactor fluid. In some such examples, the method comprises: maintaining a photo-bioreactor liquid in a substantially enclosed chamber; introducing a volume of gas; forming a bubble with a substantially non-convex shape from the volume of gas; mixing said photo-bioreactor fluid by allowing said bubble to flow through said liquid. In a further example, the method will include repeating the introducing step, the forming step and the mixing step as long as mixing of said photo-bioreactor liquid is desired. In at least some such examples, the bubble will be allowed to travel at least about one-half second before introducing another volume of gas.

In still other examples, the introducing further comprises varying the pressure on the supply of said volume of gas and/or temporarily trapping said volume of gas under a flap and/or temporarily trapping said volume of gas within a gas chamber with reverse siphon.

In still another example, a system is provided for mixing photo-bioreactor fluid. In some examples, the system comprises: means for maintaining a photo-bioreactor liquid in a substantially enclosed chamber, means for introducing a volume of gas sufficient to generate a bubble with a substantially non-convex shape. In still further examples, the system further comprises means for repeating said introducing step as long as mixing of said photo-bioreactor liquid is desired.

In at least some examples, a means is also provided for allowing said bubble to travel at least about one-half second before introducing another volume of gas.

In some specific examples, the means for introducing a volume of gas comprises a bubble generator (for example, a gas chamber with reverse siphon disposed within the chamber). Alternatively, the bubble generator comprises a gas trap disposed within the chamber (for example, a flap). In still another example, the bubble generator comprises a gas supply line capable of generating variable pressure.

In at least one example, the means for preparing an angled site comprises excavation of an earthen berm using, for example, a bulldozer or other earth moving equipment or in another example, using an angled rooftop as the angled site.

In at least one example, the means for unfurling a photo-bioreactor unit comprises manually unfurling a unit once it has been placed on the ground and in another example using a tractor to support, for example, a spool around which the unit has been rolled up.

In at least one example, the means for positioning a photo-bioreactor unit at a site comprises manually placing the unit on an earthen berm or in another example, an angled rooftop.

In at least one example, the means for connecting the lower end of a photo-bioreactor unit to a source of gas comprises manually connecting the pre-installed gas supply line that is a part of the integrated photo-bioreactor unit to a pipeline that leads to a source of carbon dioxide or other gas that can be used to support algae or other photo-microorganism growth.

In at least one example, the means for connecting the lower end and/or the upper end of a photo-bioreactor unit to a liquid source comprises manually connecting the integrated liquid fill line and/or integrated liquid fill/drain line to a pipeline that in turn leads to a source or supply of liquid medium, such as water and nutrients, needed to support microorganism growth.

In at least one example, the means for connecting the lower end of a photo-bioreactor to a liquid drain comprises manually connecting the integrated liquid drain line and/or integrated liquid fill/drain line to a pipeline that in turn leads to a liquid drain.

In at least one example, the means for rolling up a photo-bioreactor unit comprises manually rolling up the unit.

In at least one example, the means for including at least one fold comprises manually creating a fold.

In at least one example, the means for disconnecting any input or output lines to a photo-bioteactor unit comprises manually disconnecting the pre-installed gas supply line that is a part of the integrated photo-bioreactor unit from the pipeline that leads to a source of carbon dioxide or other gas that can be used to support algae or other photo-microorganism growth in a further example, manually disconnecting the integrated liquid fill line and/or liquid fill/drain line from a pipeline that leads to a source or supply of liquid medium, such as water and nutrients, needed to support microorganism growth and in a further example, manually disconnecting the integrated liquid drain line and/or liquid fill/drain line from a pipeline that leads to a liquid drain.

In at least one example, the means for maintaining a photo-bioreactor liquid in a substantially enclosed chamber comprises a thin, flexible plastic (for example, 0.1 to 200 mil thick polyvinyl chloride, polyolefin, polyethylene terephthalate, polyimide, polyurethane or similar plastics) that is non-toxic to algae but at least on one wall of the chamber transmissive to wavelengths of light needed for photosynthesis by the algae.

In at least one example, the means for introducing a volume of gas sufficient to generate a bubble having a substantially non-convex shape comprises a bubble generator. In further examples, the bubble generator comprises a gas trap disposed within the chamber. In at least one example, the gas trap comprises a flap. In a further example, the flap comprises a flexible, hinged flap. In at least one example, the bubble generator comprises a gas supply line capable of generating variable pressure. In other examples, the bubble generator comprises a gas chamber with reverse siphon disposed within the chamber.

In at least one example, the means for repeating the introducing step as long as mixing of the photo-bioreactor liquid is desired comprises an electronic control unit that will control a variable speed motor connected to a gas blower or alternatively in another example will control a control valve connected to the gas blower. In some examples, the control valve will be located upstream of the gas blower and in other examples will be located downstream of the gas blower. Periodic variation in the speed of the motor in some examples will result in periodic pressure pulses. Periodic opening and closing of the control valve will result in periodic pressure pulses in other examples. The periodic pressure pulses in some examples will influence the rate of flow and the pressure of gas in the gas supply line and will allow pulses of gas to be repeated periodically.

In at least one example, the means for repeating the introducing step as long as mixing of the photo-bioreactor liquid is desired comprises a gas supply line capable of generating variable pressure in which pressure pulses occur periodically.

In at least one example, the means for repeating the introducing step as long as mixing of the photo-bioreactor liquid is desired comprises a gas supply line with sufficient pressure and volume to fill gas traps and/or gas chambers with reverse siphons periodically with sufficient gas to periodically introduce a volume of gas sufficient to generate a bubble having a substantially non-convex shape into a photo-bioreactor chamber.

In at least one example, the means for allowing a bubble to travel at least about one-half second before introducing another volume of gas comprises an electronic control unit that will control a variable speed motor connected to a gas blower or alternatively in another example will control a control valve connected to the gas blower. In some examples, the control valve will be located upstream of the gas blower and in other examples will be located downstream of the gas blower. Periodic variation in the speed of the motor in some examples will result in periodic pressure pulses. Periodic opening and closing of the control valve will result in periodic pressure pulses in other examples. The periodic pressure pulses in some examples will influence the rate of flow and the pressure of gas in the gas supply line and will allow pulses of gas to be repeated about every one-half second or longer.

In at least one example, the means for allowing a bubble to travel at least about one-half second before introducing another volume of gas comprises a gas supply line capable of generating variable pressure in which pressure pulses occur about every one-half second or longer that are sufficient to introduce a volume of gas that generates a bubble having a substantially non-convex shape into a photo-bioreactor chamber.

In at least one example, the means for allowing a bubble to travel at least about one-half second before introducing another volume of gas comprises a gas supply line with sufficient pressure and volume to fill gas traps and/or gas chambers with reverse siphons with sufficient gas to generate a bubble having a substantially non-convex shape into a photo-bioreactor chamber about every one-half second or longer.

### BRIEF DESCRIPTION OF THE DRAWINGS

These and other attributes will become more clear upon making a thorough review and study of the following description of example embodiments, particularly when reviewed in conjunction with the drawings.

FIG. 1A is a perspective view that depicts a single chamber of the integrated unit that makes up an example of the invention.

FIG. 1B is a detail of the circled area of FIG. 1.

FIGS. 2A-2D are perspective views of several illustrative sparger designs.

FIG. 3 is a graph depicting gas pressure vs. time when the gas supply line is used to create large bubbles by generating intermittent pulses.

FIG. 4 is diagram of the components that can be used to generate intermittent pulses of gas needed to create large gas bubbles.

FIG. 5A is a side view of and example of the invention showing the flow pattern created by transit of, and the resulting non-convex shape of, a large bubble.

FIG. 5B is an illustration of how a particle within the liquid media may circulate axially and locally as a large bubble moves through a photo-bioreactor chamber.

FIG. 6 is a perspective view of a photo-bioreactor illustrating the ability to provide intermittent gas bubbles with a large distance between them.

FIGS. 7A and 7B are cross sectional views of a flap used as a gas trap intermittent large bubble generator.

FIG. 8 is a perspective view of a flap gas trap inside the lower end of a photo-bioreactor chamber.

FIG. 9 is a cross sectional view of a gas chamber reverse siphon large bubble generator.

FIG. 10A is a perspective view showing an integrated unit of the photo-bioreactor, with a plurality of chambers connected along their lengths, being unfurled or rolled up on a graded surface.

FIG. 10B is an illustration of how the number of tucks inserted during the rollup of the photo-bioreactor impacts the corresponding cross sectional shape of the chambers when unrolled and filled.

FIG. 11 is a side cross sectional view that depicts the positioning of the gas vent line, the gas supply line and the liquid fill/drain line in one example.

FIG. 12 gives a front elevation view of the photo-bioreactor in one example after unfurling and positioning along an angled berm.

### DETAILED DESCRIPTION OF EXAMPLE EMBODIMENTS

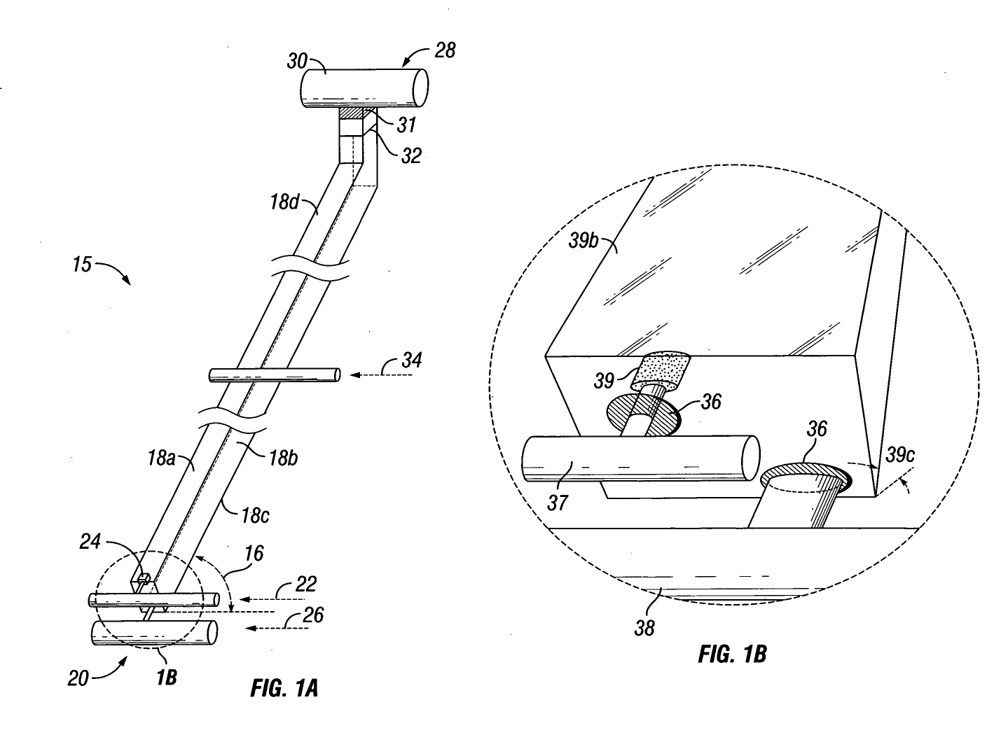

FIG. 1A shows a single chamber 15 of the integrated, elevated photo-bioreactor unit that rests at an angle 16 from level ground. The chamber in this example includes the four walls 18a-d that comprise the chamber. At least one wall—the wall that receives direct sunlight once an example of the invention is positioned correctly on an earthen berm 18d—should have a transparent surface. Other examples of the unit have chambers that have non-rectangular cross sections, including, for example, circular or oval cross sections. Chambers with circular or oval cross-sections, which may be manufactured with two sheets of material that are joined along two seams, will enjoy lower construction costs than those with four sided cross sections that require more sheets and more seams.

Positioning the photo-bioreactor on an angled earthen berm creates a lower end 20 and an upper end 28. The lower end, as depicted in FIG. 1A, is preconnected to a gas supply line 22 and sparger 24 and to a liquid fill/drain line 26. The upper end includes a preconnected gas vent line 30 with an optional demisting pad 31 to reduce carry over of the liquid growth medium into the gas vent. The optional demisting pad should be positioned above the gas-liquid interface 32. In some circumstances, the photo-bioreactor includes additional gas inlet lines 34 and spargers that provide added gas volume to the system at intermediate distances along the chamber.

The detailed view in FIG. 1B highlights the side wall reinforcements 36 at the points of connection of the gas supply 37 and liquid fill/drain 38 lines with the wall of the chamber. A sparger 39, which conducts the flow of gas into the liquid in the chamber, is included in at least one embodiment. This lower end of the chamber rests on an incline of angle 39c to level ground.

In some examples, the walls of the chamber, the gas vent line, the gas supply line, the liquid fill/drain line comprise thin, flexible plastic (for example, 0.1 to 200 mil thick polyvinyl chloride, polyolefin, polyethylene terephthalate, polyimide, polyurethane or similar plastics). The thin, flexible material may incorporate random fibers or a woven fabric as a composite, or may be bonded to a woven or non-woven fabric. These fibrous materials will enhance the strength of the flexible material composite. In at least some examples, the chamber floor, as well as the other walls, comprises flexible material reinforced with fiber, for example geo-textile, to ensure resistance to punctures and to discourage vegetation growth under the photo-bioreactor.

The seams that are formed by the chamber walls are formed in some examples by contact where the flexible plastic material meets and connects each chamber lengthwise to its adjacent chambers and at the points of connection between each of the gas/liquid lines and the walls of the chamber. Illustrative methods of joining the seams include: joined using adhesives; welded using sonar, electric, radio frequency or thermal techniques; stitched with seam sealants; and other similar, low cost techniques.

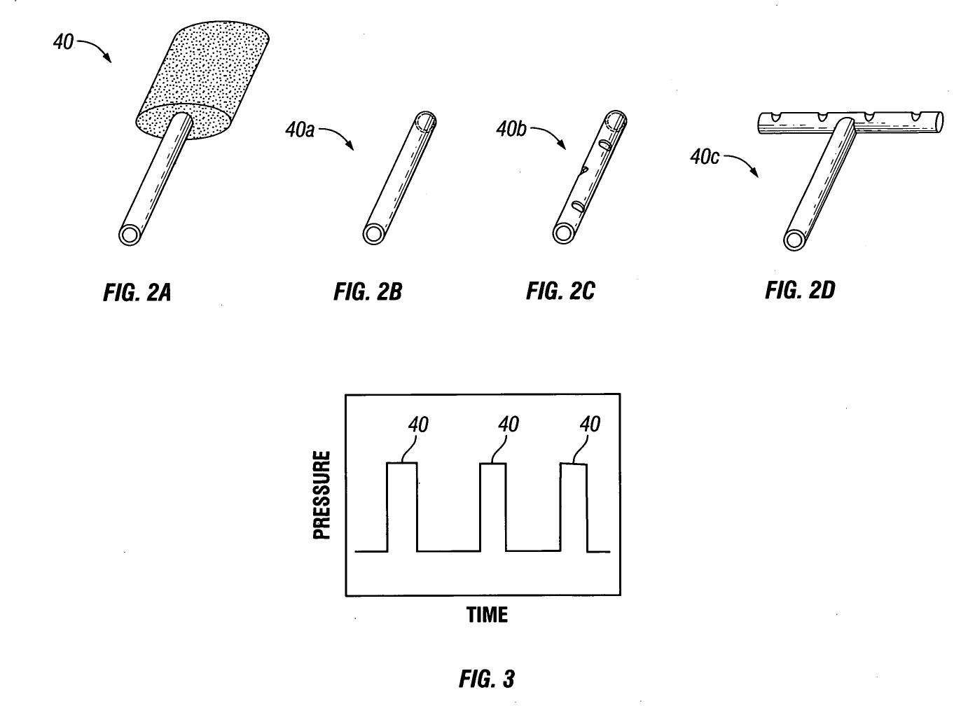

FIGS. 2A-2D show four sparger designs that can be used to disperse gas from the gas vent line into the interior of a chamber. In at least some examples, an end of the gas supply line that disperses gas is connected, FIG. 2A, to permeable stone 40, or includes in FIG. 2B a single exhaust port 40a. The length of the sparger in 2B may be varied, and includes very short lengths. A further example, FIG. 2C, includes an opening at the end, and multiple openings along the pipe 40b. A further example, FIG. 2D, is configured as a “T” with openings along the crossbar portion of the pipe 40c. Instead of a sparger, the gas may enter the bioreactor chamber from the gas supply line through an open port aligned with the chamber wall.

The bubbles used to induce mixing will be created in some examples by intermittently pulsing the gas input. As illustrated in FIG. 3, this methodology will result in intermittent gas pressure peaks 40, as illustrated in FIG. 3's graph of gas pressure against time.

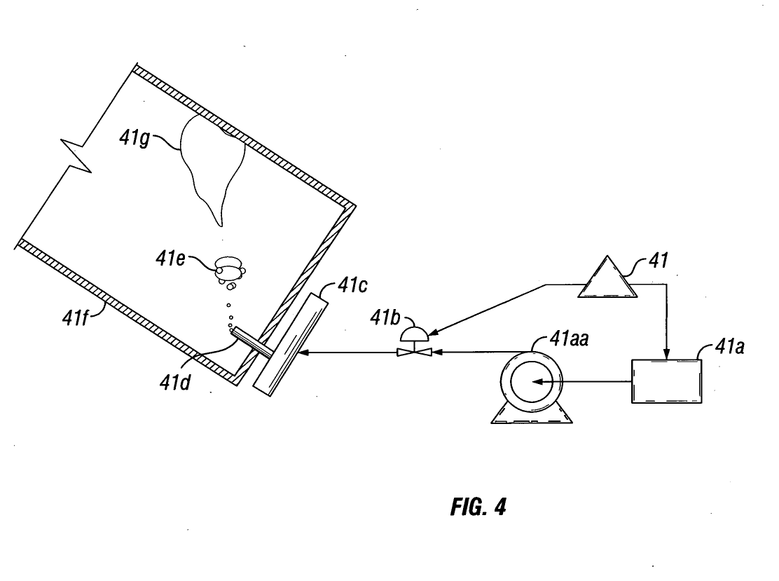

As illustrated in FIG. 4, the pressure peaks will be induced in some examples by action of an electronic control unit 41. In some examples, the electronic control unit will control a variable speed motor 41a connected to a gas blower 41aa. Alternatively, the electronic control unit will operate the control valve 41b. In some embodiments, the control valve will be located upstream of the gas blower. Periodic variation in the speed of the motor results in periodic pressure pulses. Periodic opening and closing of the control valve also results in periodic pressure pulses. The periodic pressure pulses will influence the rate of flow and the pressure of gas in the gas supply line 41c and will allow pulses of gas to be repeated periodically, including about every one-half second or longer. The sparger 41d, or in some examples an opening in the photo-bioreactor chamber wall, that introduces gas from the gas supply line when the gas pressure is being pulsed, introduces one or more bubbles 41e into the liquid in the interior of the photo-bioreactor chamber 41f that rise and coalesce to form fewer, larger bubbles 41g.

In other examples, the means for allowing a bubble to travel a specified length of time, including for example, at least about one-half second, before introducing another volume of gas comprises a gas supply line capable of generating variable pressure in which pressure pulses occur about every one-half second, or longer, that are sufficient to introduce a volume of gas that generates a bubble having a substantially non-convex shape into a photo-bioreactor chamber.

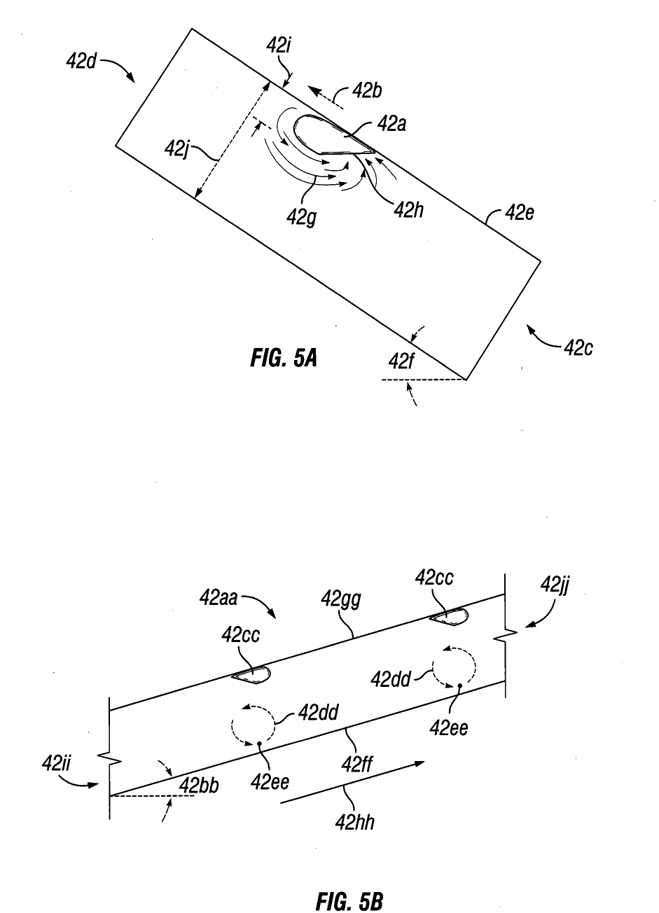

As the bubbles rise, liquid is displaced around the bubble, causing local, radial flow. In some examples, at a particular minimum bubble volume, the local displacement of liquid transforms from laminar to turbulent flow, vastly enhancing mixing. In many examples, the trailing edge of the bubble is non-convex. The optimum bubble volume depends on the viscosity of the liquid, degree of incline, gas and liquid density and interfacial tension between the liquid and gas. FIG. 5A illustrates how a bubble 42a moves 42b from the lower end 42c to the upper end 42d of the photo-bioreactor chamber 42e, in many examples, that rests at an angle 42f from level ground. As the bubble transits, it causes liquid flow around the bubble 42g. When the bubble is of sufficient size, it will display non-convex geometry (for example, a flat trailing edge 42h). Enhanced mixing occurs when the width of the bubble 42i is at least five percent (5%) of the width 42j of the chamber's cross-section. Bubble-induced mixing with radial flows tends to increase as bubble size increases. In most examples bubble size is limited to reduce or avoid slug flow (i.e. where the gas phase completely displaces the liquid phase across the entire cross section). Slug flow greatly disturbs the liquid and is energy intensive.

As illustrated by FIG. 5B, in a photo-bioreactor chamber 42aa resting at an angle 42bb from level ground, displaced liquid flow in the vicinity of the large bubbles 42cc in a photo-bioreactor chamber creates a local axial movement 42dd that drives particles 42ee in the liquid, such as photo-microorganisms, away from the bottom 42ff of the photo-bioreactor chamber toward the top, transparent wall 42gg that receives sunlight, with the flow in a randomized circular motion 42dd. The bubbles 42cc move 42hh from the lower end 42ii to the upper end of the chamber 42jj

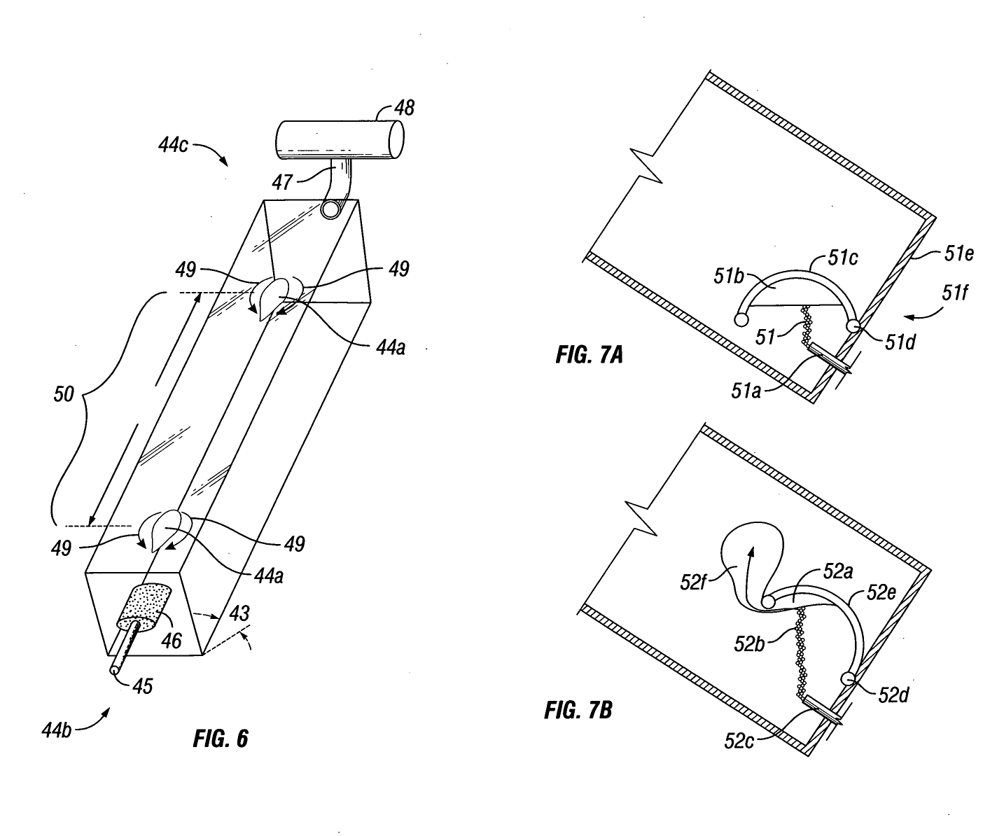

FIG. 6 illustrates an example of a single photo-bioreactor chamber positioned on a graded surface of angle 43 from ground level. The bubble generator—in this case the gas supply line itself that is being pulsed—creates rising gas bubbles 44a that travel from the lower end of the chamber 44b, which has a connection 45 to the gas supply line and optional stone sparger 46, to the upper end 44c, which is connected 47 to the gas vent line 48. The flow around the bubbles 49 mixes the liquid inside the chamber. The frequency of bubble generation, and corresponding distance between the bubbles 50, will be timed in some examples to optimize mixing by varying the frequency based on the photo-microorganism's natural photo-saturation or chemical relaxation cycle time.

Another embodiment includes a gas trap, disposed within a chamber, that is attached to a hinge on the wall of the chamber near the opening of the sparger. In some embodiments, the hinge and the sparger, or in some examples the opening to the gas supply line, will be on the floor of the chamber. In FIG. 7A, gas 51 from the sparger 51a creates an air bubble 51b inside the flexible flap 51c (i.e. gas trap), which is connected via a hinge 51d to the photo-bioreactor chamber wall 51e on the lower end of the chamber 51f. In FIG. 7B, the air bubble 52a becomes large enough, as it traps gas 52b from the sparger 52c, to exert buoyancy sufficient to cause the hinge 52d to open and to thereby cause the flexible flap 52e to release a large gas bubble 52f.

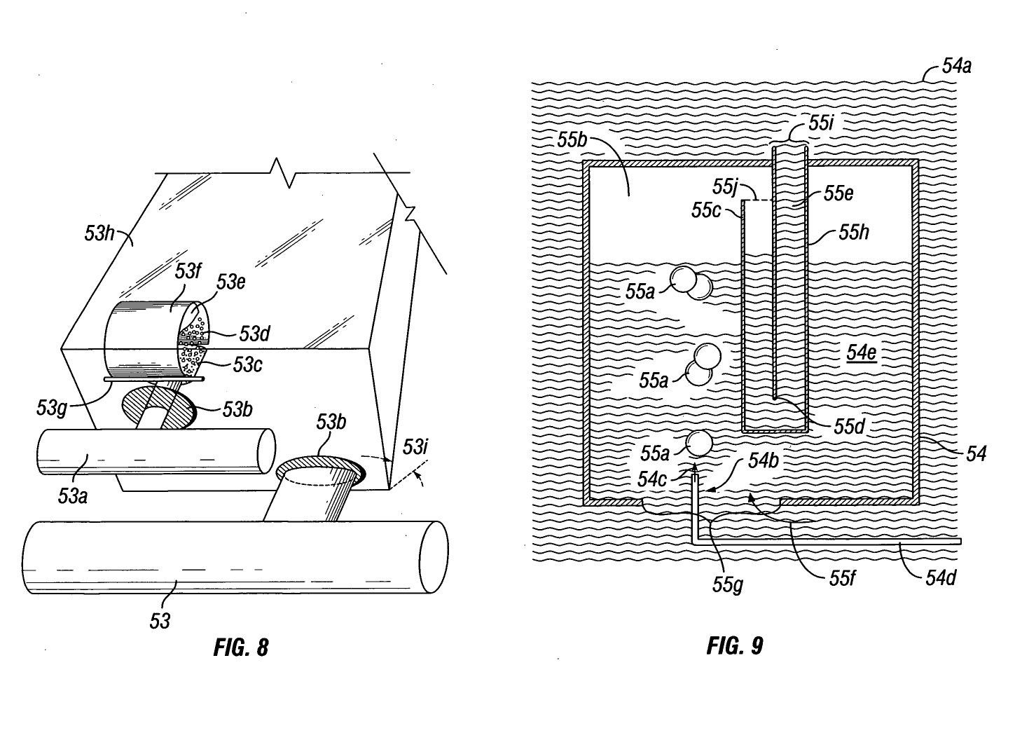

FIG. 8 illustrates how the flexible flap gas trap will be positioned in at least some examples at the lower end of a photo-bioreactor chamber. The lower end of the chamber is connected to a liquid fill/drain line 53 and gas supply line 53a and has reinforced plastic at those connections 53b. The sparger 53c generates small bubbles 53d that form a large air bubble 53e trapped by the flexible flap gas trap 53f. Once sufficient gas accumulates, the flap swings open on its hinge 53g releasing a large air bubble that floats toward the transparent top of the chamber 53h. The photo-bioreactor chamber rests at an angle 53i to level ground.

Alternately, a gas chamber with reverse siphon, disposed within a photo-bioreactor chamber, will be used to generate large bubbles. Several references disclose alternative siphon designs that will generate, large bubbles (U.S. Pat. No. 2,717,774 Obma; U.S. Pat. No. 3,246,761 Bryan et al.; U.S. Pat. No. 3,592,450 Rippon; U.S. Pat. No. 3,628,775 McConnell et al.; U.S. Pat. No. 4,169,873 Lipert; U.S. Pat. No. 4,187,263 Lipert; U.S. Pat. No. 4,293,506 Lipert; U.S. Pat. No. 4,337,152 Lynch; U.S. Pat. No. 4,356,131 Lipert; U.S. Pat. No. 4,439,316 Kozima et al.); all of the preceding are incorporated herein by reference for all purposes.

FIG. 9 illustrates an example of a gas chamber reverse siphon that is disposed within a photo-bioreactor chamber and will generate large bubbles. The chamber 54 is submerged in liquid 54a within the photo-bioreactor chamber. The sparger 54b releases gas 54c from the gas supply line 54d into the interior of the gas chamber 54e in the form of small gas bubbles 55a. The bubbles enter the interior of the gas chamber 54e, gradually filling the top of the chamber 55b and the reverse siphon leg 55c and displacing liquid. Once the liquid level is low enough to reach the bottom of the reverse siphon leg 55d, gas pushes out the liquid located inside the siphon tube 55e. Once this occurs, liquid flows 55f in from the opening 55g in the bottom of the chamber and pushes the gas at the top of the chamber through the reverse siphon tube 55c and the siphon tube 55h. This creates a large bubble by pushing the gas collected in the top of the chamber rapidly out the siphon tube opening 55i. The liquid level in the gas chamber rises rapidly to the top entry of the reverse siphon 55j, and then refills the reverse siphon tube 55c and siphon tube 55h. When the siphon tubes are filled with liquid, further movement stops, and the cycle repeats with gas flow into the gas chamber.

In some examples, a gas supply line with sufficient pressure and volume will be used to fill bubble generators such as gas traps and/or gas chambers with reverse siphons periodically, including for example about every one-half second, or longer, with sufficient gas to periodically (for example about every one-half second, or longer), introduce a volume of gas sufficient to generate a bubble having a substantially non-convex shape into a photo-bioreactor chamber.

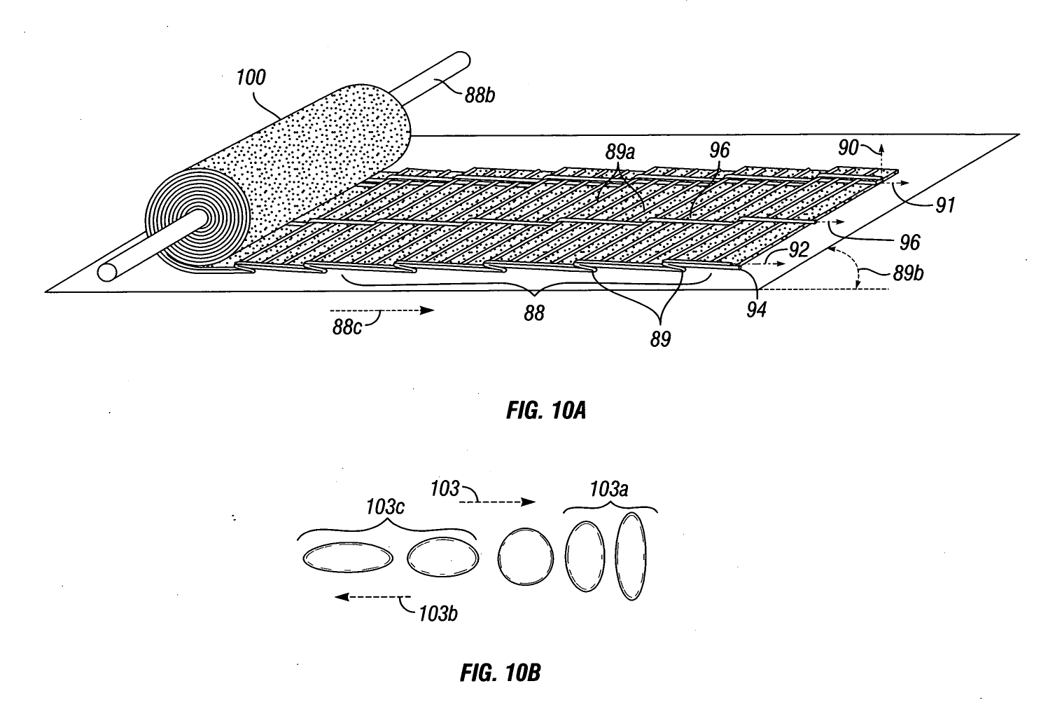

An integrated unit of some examples includes a plurality of photo-bioreactor chambers 88 flexibly joined along their lengths, as illustrated in FIG. 10A. After the flexible photo-bioreactor has been fabricated at the factory, it is rolled up on a long bar or spool 88b for transportation. During the rolling process 88c at the factory, the flexible material is repeatedly folded or tucked, with a typical practice of one fold approximately every fourth chamber 89 so that, when filled with liquid medium, the unit will fill up without placing undue lateral strain, on the joints and the flexible material comprising the individual chambers 89a. The rolling up will be accomplished manually or via machine (for example a tractor supporting the spool on which the photo-bioreactor unit has been rolled up). The folds will be added manually in some embodiments. Excess strain will potentially damage one or more chambers.

The photo-bioreactor is unfurled in many examples on a graded ground surface prepared at angle 89b that optimizes exposure to solar radiation, gas flow within the chambers, and fill/drain hydraulics. The photo-bioreactor is aligned on the surface so that the ends of the chambers that are pre-joined to a common gas vent line 90 with demisting pad 91 become the elevated or upper end. The end of the chambers pre-joined to a common gas supply line 92 and a common liquid fill/drain line 94 becomes the lower end. This embodiment also includes a second or more, optional gas supply line 96. The bottom of this unit has been reinforced with geo-textile 100. The size and weight of the unit should permit the unit to be manually rolled up, both in the field or in the factory, or manually unfurled and positioned. Alternatively, available mechanical devices, such as a tractor, can be used to roll up or unfurl and position the unit.

As FIG. 10B illustrates, increasing the number of folds 103 will decrease the lateral stress on the flexible material and joints and upon being filled with liquid creates chambers with approximately vertical oval cross sections 103a while decreasing the number of folds 103b will generate greater lateral stress and create approximately horizontal oval cross sections 103c. Two-sided chambers with circular, rather than four sided, cross sections will experience greater lateral stress and thus have greater need for the aforementioned folds.

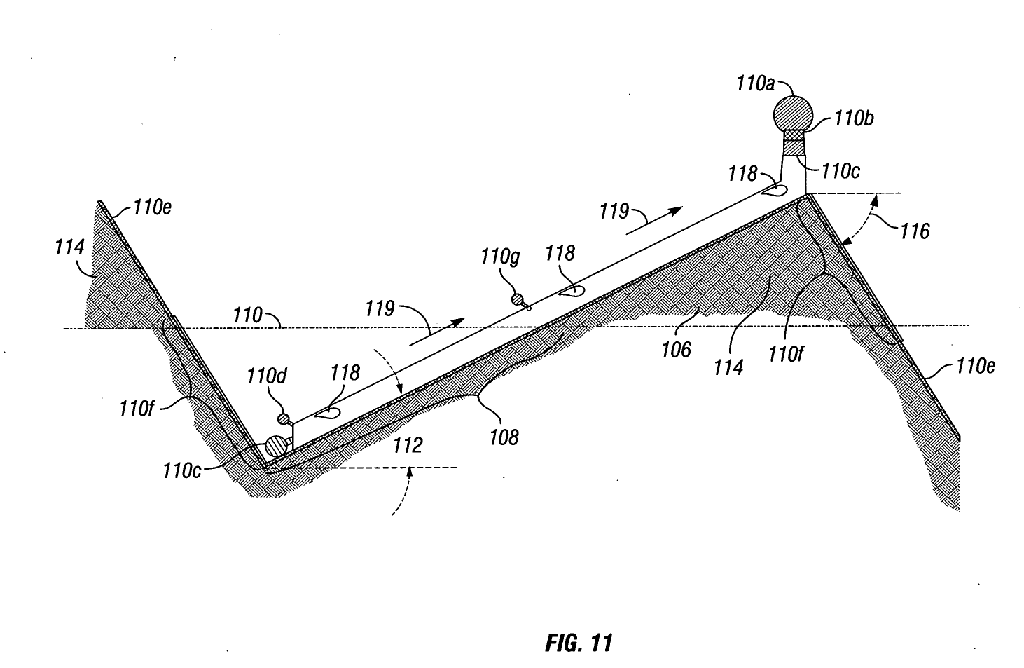

FIG. 11 illustrates a side, cross-sectional view of and example of a photo-bioreactor unit after it has been unfurled and positioned on an angled earthen berm 106. In this example, the earthen berm includes a portion 108 that lies below the original ground level 110. The example shown is positioned with the gas vent line 110a and demister 110b (and accompanying liquid/gas interface 110c) on the upper end of the berm and gas supply lines 110d and liquid fill/drain line 110e on the lower end of the berm. Optionally, a geotextile, in some embodiments, will be laminated or glued to the bottom of the chambers to enhance resistance to punctures and to discourage vegetation growth under the chambers. The geotextile will extend flaps 110f, in some examples, beyond the ends of the chambers that will lie over the uncovered face of the earthen berm to reduce erosion and unwanted vegetation growth that could shade the photo-bioreactor. In other examples, the liquid fill line will be positioned on the section of the photo-bioreactor unit that lies on the upper end of the berm and the liquid drain line will be positioned on the section of the photo-bioreactor unit that lies on the lower end of the berm. In some examples, as shown in FIG. 11, there is an optional, additional gas supply line 110g. In further examples, parallel rows of chambers and berms are installed on a field, geotextile fabric flaps from adjacent units will be overlapped and possibly joined or staked, to create a continuous barrier to erosion and unwanted vegetation growth across the field. The angle 112 between the original ground level 110 and the angled surface of the berm will range in some embodiments from 10 to 70 degrees. The optimal angle depends on latitude, which affects the best angle to maximize incoming sunlight, and the need to optimize the use of gas bubbles to maximize mixing hydraulics.

In some situations, earth from the excavation of the berm will be used as fill 114 to create a portion of the berm that rises above ground level. The angle 116 between the original ground level and the surface of the berm that forms the backside of the berm—away from the surface on which the photo-bioreactor rests—ranges, in some example embodiments, from 0 to 150 degrees depending on ground composition, resistance to slippage and the underlying slope of the original ground. The large bubbles 118 with a non-convex trailing edge move 119 toward the upper end of the unit from the lower end.

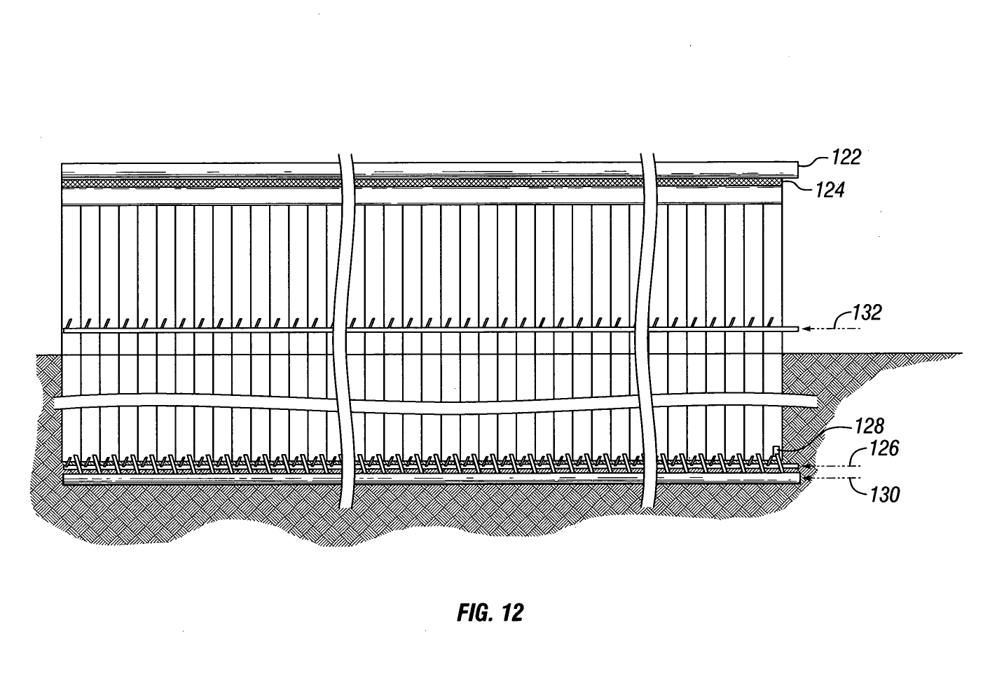

After setup and from a front elevation perspective, as in FIG. 12, the photo-bioreactor appears to be a long series of repeating chambers with the gas vent line 122 (with optional demisting pad 124) connected to the upper end of each chamber. The gas inlet line 126 (with optional sparger 128) and liquid fill/drain line 130 are connected to the lower end of each chamber. This example also has a second, optional gas inlet line 132.

Those skilled in the art will appreciate that adaptations and modifications of the example embodiments can be employed without departing from the scope and spirit of the invention. Nothing in this document is intended as a limitation on the scope of the claims provided below.

What is claimed is:

1. A photo-bioreactor comprising:
a plurality of substantially enclosed, flexibly connected, parallel chambers, at least some of said chambers comprising:
an upper end connected to at least one flexible gas vent line;
a lower end connected to at least one flexible gas supply line;
said lower end connected to at least one flexible liquid fill/drain line and/or said upper end connected to a flexible liquid fill line and said lower end connected to a flexible liquid drain line;
at least one transparent wall to each chamber;
at least some of said plurality of chambers, said connections, said gas vent line, said gas supply line, said liquid fill line, said liquid drain line and/or said liquid fill/drain line being comprised of thin, flexible materials.

2. The photo-bioreactor of claim 1 in which at least some of said thin, flexible materials comprise fibrous reinforcement.

3. The photo-bioreactor of claim 1 further comprising a bubble generator.

4. The photo-bioreactor of claim 3 in which said bubble generator further comprises a gas trap disposed within at least some of said chambers.

5. The photo-bioreactor of claim 4 in which said gas trap further comprises a flap.

6. The photo-bioreactor of claim 3 in which said bubble generator further comprises a gas chamber with reverse siphon disposed within at least some of said chambers.

7. The photo-bioreactor of claim 3 in which said bubble generator further comprises a gas supply line capable of generating variable pressure.

8. A method of setting up a photo-bioreactor, the method comprising the steps of:
preparing an angled site;
unfurling said photo-bioreactor on said site;
positioning said photo-bioreactor on said site so that there is an upper end that is elevated with respect to the lower end.

9. The method of claim 8 further comprising the step of connecting said lower end to a source of gas.

10. The method of claim 8 further comprising the step of connecting said lower end and/or said upper end to a source of liquid.

11. The method of claim 8 further comprising the step of connecting said lower end to a liquid drain.

12. A system for setting up a photo-bioreactor, the system comprising
means for preparing an angled site;
means for unfurling said photo-bioreactor;
means for positioning said photo-bioreactor on said site so that there is an upper end that is elevated with respect to the lower end.

13. The system of claim 12 further comprising a means for connecting said lower end to a source of gas.

14. The system of claim 12 further comprising a means for connecting said lower end and/or said upper end to a source of liquid.

15. The system of claim 12 further comprising a means for connecting said lower end to a liquid drain.

16. A method of handling a photo-bioreactor comprising the steps of
rolling up said photo-bioreactor;
including at least one fold.

17. A system of handling a photo-bioreactor comprising:
means for rolling up said photo-bioreactor;
means for including at least one fold.

18. A method of disassembling a photo-bioreactor comprising the steps of:
disconnecting any input and/or output lines to said photo-bioreactor;
rolling up said photo-bioreactor.

19. The method of claim 18 in which said step of rolling up said photo-bioreactor comprises including at least one fold.

20. A system of disassembling a photo-bioreactor comprising
means for disconnecting any input or output lines to said photo-bioreactor;
means for rolling up said photo-bioreactor.

21. The system of claim 20 in which said means for rolling up said photo-bioreactor comprises means for including at least one fold.

22. A method of mixing photo-bioreactor fluid, the method comprising the following steps:
maintaining a photo-bioreactor liquid in a substantially enclosed chamber;
introducing a volume of gas;
forming a bubble with a substantially non-convex shape from the volume of gas;
mixing said photo-bioreactor fluid by allowing said bubble to flow through said liquid.

23. The method of claim 22 further comprising repeating said introducing step, said forming step and said mixing step as long as mixing of said photo-bioreactor liquid is desired.

24. The method of claim 22 further comprising allowing said bubble to travel at least about one-half second before introducing another volume of gas.

25. The method of claim 22 in which said introducing step further comprises varying the pressure on the supply of said volume of gas.

26. The method of claim 22 in which said introducing step further comprises temporarily trapping said volume of gas under a flap.

27. The method of claim 22 in which said introducing step further comprises temporarily trapping said volume of gas within a gas chamber with reverse siphon.

28. A system of mixing photo-bioreactor fluid, the system comprising the following elements:
means for maintaining a photo-bioreactor liquid in a substantially enclosed chamber;
means for introducing a volume of gas sufficient to generate a bubble with a substantially non-convex shape;

29. The system of claim 28 further comprising means for repeating said introducing step as long as mixing of said photo-bioreactor liquid is desired.

30. The system of claim 28 further comprising means for allowing said bubble to travel at least about one-half second before introducing another volume of gas.

31. The system of claim 28 in which said means for introducing a volume of gas comprises a bubble generator.

32. The system of claim 31 in which said bubble generator further comprises a gas chamber with reverse siphon disposed within said chamber.

33. The system of claim 31 in which said bubble generator further comprises a gas trap disposed within said chamber.

34. The system of claim 33 in which said gas trap further comprises a flap.

35. The system of claim 31 in which said bubble generator further comprises a gas supply line capable of generating variable pressure.
